(19) United States
(12) Patent Application Publication
(10) Pub. No.: US 2019/0325725 A1
(43) Pub. Date: Oct. 24, 2019

(54) SYSTEM FOR MONITORING A PERSON WITHIN A RESIDENCE

(71) Applicant: NOVIA SEARCH, Florange (FR)

(72) Inventors: Regis CIRE, Differdange (LU); Mathieu MULLER, Valleroy (FR)

(21) Appl. No.: 16/312,527

(22) PCT Filed: Jul. 3, 2017
(86) PCT No.: PCT/FR17/51795

§ 371 Date: Dec. 21, 2018

(30) Foreign Application Priority Data
Jul. 5, 2016 (FR) 1656420

(51) Int. Cl. G08B 21/02; G16H 50/20; G16H 40/63; G08B 21/18

(52) U.S. Cl. CPC G08B 21/02; G08B 21/182; G16H 40/63; G16H 50/20

(57) ABSTRACT

The system for monitoring a person includes primary and secondary detection devices equipped with devices for sending measured data through a communication network; and a centralization device provided with a receiver for measured data and a processor for the received measured data. The processor includes a software application executed in a learning mode to store the received measured data and to determine a monitoring profile of the person and executed in a comparative mode to compare the received measured data with stored data and to emit an alert signal if one of the received measured data deviates from a defined interval around the monitoring profile.

### CROSS-REFERENCE TO RELATED APPLICATIONS

See Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

The present invention falls within the field of monitoring and assistance for persons.

The invention finds a preferred, but in no way limiting, application in the monitoring of at-home activity for an elderly person, a patient, for example in outpatient medicine, or any person in a state of physical weakness or an unstable psychological state. Such monitoring should be automatic in order to provide assistance to said person in case of need.

### 2. Description of Related Art Including Information Disclosed Under 37 CFR 1.97 and 37 CFR 1.98

Today, the increased life expectancy and the increased number of elderly people, as well as the evolution in outpatient medicine, compared with the limited development of care infrastructure, whether hospital establishments or related facilities, such as retirement homes, have caused an increase in people whose follow-up is provided at home when possible. Without close family, it is difficult to have constant human monitoring in each person's home.

In order to provide this monitoring automatically, many different devices exist. For example, there are connected portable objects, in the form of a pendant or bracelet, equipped with an alert button allowing its wearer, in case of difficulty, to notify emergency services for intervention. Other devices, such as bracelets, incorporate an accelerometer making it possible to detect a fall and send a signal in order to alert the emergency services.

A first drawback of the state of the art lies in the interaction between the person and the device he is wearing. If consciousness is lost, in particular without a fall, most of the known devices do not make it possible to emit an alert. Furthermore, they require the person to be able to remember to wear said device systematically, which may not always be the case, for example when grooming oneself. Indeed, even with monitoring, many household accidents occur in the bathroom, often due to slippery surfaces.

Additionally, the major shortcoming lies in the radical shift in a person's behavior, for example when struck with dementia caused by a loss in memory, like in the case of Alzheimer's disease. This disease may then result in a deterioration in daily hygiene, both bodily and household, undernourishment, becoming lost, etc.

### BRIEF SUMMARY OF THE INVENTION

The present invention starts from this observation and the need to monitor a person's activity, in order to detect a change in his behavior that would correspond to a danger to himself, then no longer requiring at-home monitoring, but placing him in a supportive environment, or hospitalizing him.

To that end, the invention aims to offset the drawbacks of the state of the art by proposing a system for monitoring the activity of a person capable, on the one hand, of learning the typical behavior of said person, and on the other hand, of detecting any significant change in such behavior, requiring providing that person with assistance.

The invention therefore relates to such a system for monitoring a person within at least one room, which comprises:
at least separate primary and secondary detection means arranged inside said room, each detection means measuring data within said room and being equipped with means for sending data measured through a communication network; and
centralization means provided on the one hand with means for receiving said data measured by each detection means, and on the other hand means for processing the received measured data,

characterized in that said processing means consist of at least one software application executed
on the one hand, in a learning mode over a first predefined duration, storing said measured and received data, so as to determine at least one monitoring profile of said person within each room, and
on the other hand, later, in a comparative mode over a second duration, comparing said measured and received data with said stored data, so as to emit an alert signal if at least one of said data measured and received during said second duration deviates from an interval defined around said monitoring profile.

Thus, such a system first records data corresponding to the living habits of a person, by visited room, and secondly compares them with data measured in real-time. It is then possible to determine a living profile of each monitored person and, for each data type, to recognize a behavioral anomaly of said person, in order to alert the emergency services.

Non-limitingly, a measured data type consists of detecting movement, making it possible to determine time ranges where the person typically visits one room or another.

To that end, the monitoring system according to the invention is further characterized by the fact that said primary detection means comprise at least one movement detector, determining the mobile presence of a person within said room, and by the fact that said processing means apply at least one timestamp to each determined presence, so as to define at least one presence time range of said person moving within said room.

According to another advantageous feature of the invention, in this case, it is also provided that, in learning mode, said processing means perform at least one weighted average of at least two time ranges defined successively, so as to determine, within said monitoring profile, at least one normal presence period within said room.

Alternatively, it is possible to apply a preferred, but in no way limiting, monitoring of the bathroom. To that end, the invention monitors the fluctuations of the environment within this room, namely the humidity and the temperature, so as to determine the usage habits of the bathroom by the person.

To that end, the present invention provides that the secondary detection means of the monitoring system according to the invention comprise at least one detector for the environmental conditions of said room, such as at least the temperature and/or the hygrometry, while said processing means apply at least one timestamp to the detected environmental conditions, so as to determine normal environmental conditions recorded within said monitoring profile.

In this case, according to the invention, in the comparative mode, said processing means perform a comparison of the detected environmental conditions with said recorded normal environmental conditions.

### BRIEF DESCRIPTION OF THE SEVERAL VIEWS OF THE DRAWINGS

Other features and advantages of the invention will emerge from the following detailed description of non-limiting embodiments of the invention, in reference to the appended figures.

FIG. 1 is a schematic view showing an example architecture according to one preferred, but non-limiting, operating mode of the system according to the invention, showing the monitoring of a person in a bathroom.

FIG. 2 is a graph illustration transcribing an example of the evolution of the environmental conditions measured within the bathroom during use by the person, showing the curves of the hygrometry and the temperature over time.

FIG. 3 shows a graph illustration transcribing example movement detection within two different rooms over time.

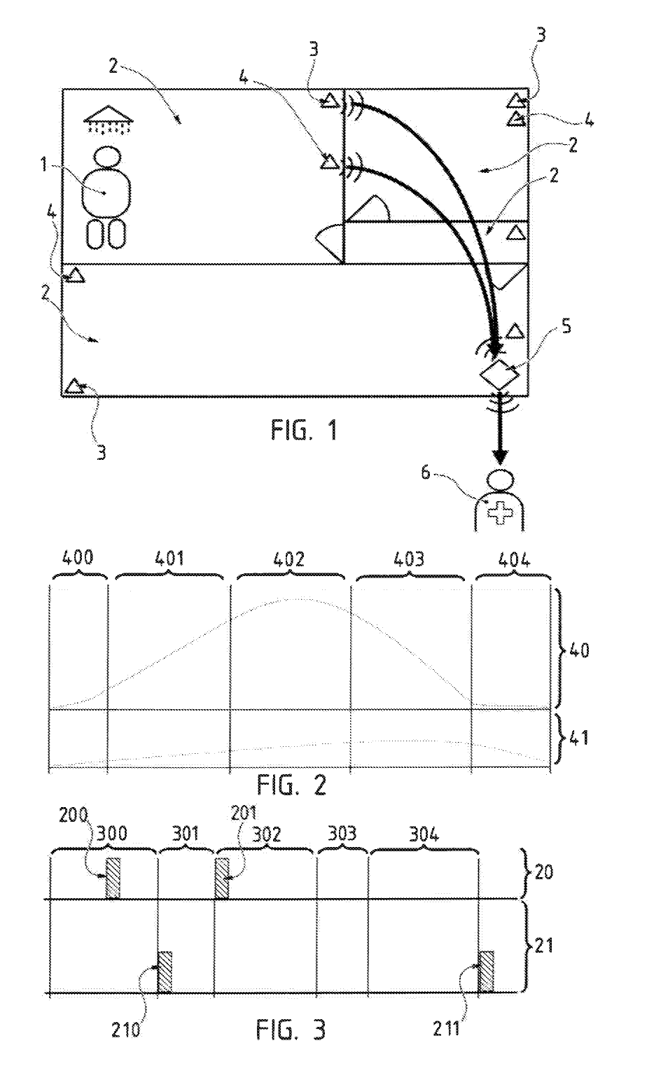

### DETAILED DESCRIPTION OF THE INVENTION

The present invention relates to a system for monitoring a person 1 within at least one room 2.

Preferably, said system makes it possible to monitor said person 1 separately in several different rooms 2, within a same home.

To that end, said system comprises at least separate primary 3 and secondary 4 detection means, namely differentiated from one another. Said primary 3 and secondary 4 means make it possible to detect different data. Other complementary detection means may be integrated into the system, to bring the detection of data to three, four or more.

Said detection means 3, 4 are arranged inside said room 2. In other words, each room 2 is equipped with appropriate detection means, based on the use made by the person 1 of said room 2 in question. To that end, each detection means 3, 4 comprises one or more sensors that measure data within said room 2. Non-limitingly, data measured by the detection means 3, 4 can be a movement or presence, the hygrometry, the temperature, the turning on or the turning off of electrical and/or electronic means, the opening and/or the closing of doors, windows and/or drawers.

The detection means 3, 4 are equipped with means for sending data measured through a communication network. Such a network can be inside the home and can be provided to be wired, but preferably wireless, namely using radio waves. The use of a wireless network makes it possible to make the installation of the detection means 3, 4 easy in each room 2.

Likewise, the detection means 3, 4 can be supplied with electricity by being connected to the electrical grid of the room 2 of the home and/or autonomously, via internal batteries, which may or may not be rechargeable.

In the alternative, the detection means 3, 4 can be electrically connected to the network of the home and use said network to send the recorded data, in particular by using a powerline communication (PLC).

The monitoring system also comprises centralization means 5. The latter can be located within the same room 2 and/or in a different room. In particular, in the context of monitoring all of the rooms of a home, the centralization means 5 are located within a room, which in turn is equipped with primary 3 and secondary 4 detection means, while each of the other rooms is only equipped with its own primary 3 and secondary 4 means.

On the one hand, said centralization means are provided with means for receiving said data measured by each detection means 3, 4. Said receiving means are then connected to the detection means 3, 4 by using the aforementioned communication network inside the home. In short, the centralization means 5 connect the detection means 3, 4 of each room 2 and recover the data that they measure.

Furthermore, the connection of the detection means 3, 4 with the centralization means 5 can be done by a configuration step during the installation of the monitoring system inside the rooms 2 of the home. A dedicated interface can be considered, accessible via said centralization means 5, in particular by means of an interface accessible via a browser executed on a separate portable terminal.

On the other hand, said centralization means 5 comprise means for processing the received measured data. Said processing means consist of at least one executed software application. Such an application makes it possible to store locally, in particular within a database, each type of data measured by each type of sensor of the detection means 3, 4. Furthermore, said application makes it possible to process, separately or in a combined manner, said already stored data, as well as the data measured and communicated in real-time.

Essentially, on the one hand, the execution of the application of the processing means is done in a learning mode over a first predefined duration.

During this first time period, the system stores said measured and received data through the processing means, so as to determine at least one monitoring profile for said person 1 within each room 2.

In short, the invention inventories, over time, through the detection by its sensors, each datum relative to the behaviors and habits of the person 1 to be monitored, within at least one room 2 deemed important, such as the bathroom, up to each room 2 of the home.

Thus, the invention records different actions performed by the monitored person 1, such as the duration of his presence in a room 2, his comings and goings, as well as some of his actions.

The first duration can be configured over a time range of from one or two days up to several days, a week or several weeks. This first duration is defined based on said person, the rooms of the home.

It will be noted that this first duration may be defined for each room 2. In other words, as an example, a first duration may be assigned to each room based on its use. For example, one or two days suffice to define the typical behavior of a person within a kitchen, his bedroom or a bathroom, based on his habits, like in particular to define mealtimes, the times he gets up or goes to bed and grooming times, whereas other rooms such as the living room, toilets or others may fluctuate considerably from one day to the next and therefore take a longer initial duration to record measured data.

Furthermore, during this learning period of the system according to the invention, additional human monitoring may be provided to said person 1, in order to avoid any accident.

On the other hand, later, the execution of the software application of the processing means is done in a comparative mode over a second duration.

The second duration is therefore later than the first duration. It will be noted that the second duration can be indefinite, unlike the first duration.

During this second duration, the primary 3 and secondary 4 detection means continue to measure and send data to the centralization means 5. Said application compares said measured and received data with said stored data. In other words, the application receives new data detected in real-time during the second duration in order to compare them with the corresponding data, recorded earlier, during the first duration. The aim is to detect any unusual behavior of the person 1 during the second duration, relative to his habits recorded earlier. In this case, if at least one of said data measured and received during said second duration deviates from an interval defined around said monitoring profile, then the invention emits an alert signal.

In a related manner, an alert to the outside can also be emitted if a malfunction of one of the elements of the system is detected, for example the loss or poor reception of the signal from a sensor.

Furthermore, the centralization means 5 may be equipped with means suitable for allowing the sending of such a signal in order to alert said person 1 as well as an outside operator 6, such as a health service. Then, in order to notify the person 1 on site, the centralization means 5 may comprise visual alarm means, in the form of indicators displaying different colors and/or shapes, and/or audio alarm means, with speakers.

In a related manner, said speakers may also emit audio indications, intended for said person 1, in order to notify him, in case of problem, or simply in case of reminder, for example regarding a meal not consumed at an observed time. In particular, said audio indications may consist of emitting prerecorded sounds in the form of voice synthesis, orally describing, at an adjustable volume, the warning or the alert in question, in order to question the monitored person 1.

As previously mentioned, the case of alert, said signal may be emitted toward the outside of the home. To that end, the centralization means 5 may be connected to a communication network, such as the telephone network, in a wired or wireless manner, by using cellular telephone communication technologies.

Furthermore, said emitted signal may contain information, in the form of a report, outlining the reason for the alert.

To that end, in the context of an installation, said system may be equipped with primary detection means 3 comprising at least one movement detector. The latter determines the mobile presence of a person 1 within said room 2. Thus, each part 2 may be equipped with its movement detector, making it possible to determine the presence and above all the movements of the monitored person 1 from one room 2 to another and/or within each room 2.

Said processing movements then apply at least one timestamp to each determined presence, so as to define at least one presence time range of said moving person 1 within said room 2. In other words, by grouping together all of the presence time ranges in one or several rooms 2, the invention makes it possible to determine the movement habits of the person within his home. Once the first duration has expired, the learning mode has therefore defined the occupancy time ranges of said monitored room 2, as well as more depth for each of the rooms 2.

More specifically, in learning mode, said processing means can perform at least one weighted average of at least two time ranges defined successively, so as to determine, within said monitoring profile, at least one normal presence period of the person 1 within said room 2. Preferably, for each room 2, the processing means define one or several occupancy time ranges, and, depending on the time ranges, in particular located at shared times, namely around the same time, the invention determines a normal usage duration around said time. In case of significant excess, by several minutes of the normal time range, the system may then consider that there is a problem and emit said alert signal.

As an example, in reference to FIG. 3, the monitoring of the movements within two rooms is modeled. This graph shows, over time along the x-axis, the detection of movements within a first room 20 in the upper part and a second room 21 in the lower part. Several time ranges 300 to 304 have been determined. As shown, first of all, movements are detected within the first room 20, shown by a first crosshatched rectangle 200, during the first time range 300.

Furthermore, it is noted that the detection 200 does not take place at the beginning, but during the first period 300, within the time range previously determined. In short, the person 1 enters the room as usual, but with a slight delay, without deviating from his normal behavior.

Next, during another time range 301, a detection takes place in the second room 21, shown by a second rectangle 210. Once the detection 210 within the second room 21 takes place, then the system takes it into account and stops the monitoring over the typical time range 300 of the first room 21. This monitoring restarts upon the detection 201, during a following period 302. One can then see that no movement is detected for the rest of this period 303, until reaching a limited duration, at the end of which, the system having previously learned the average duration that the person 1 spends in this room, but there is potentially a problem in his behavior. An alert mode can then be triggered, in period 303, warning said person 1 with said signal. If no intervention by the person 1 takes place at the end of said period 303, then the system goes to a following period 304, sending a signal to the outside operator 6.

However, as shown in FIG. 3, if another movement 211 is detected in the other room 21, then the system cancels the monitoring of the first room 20 and the sending of the alert signal to the inside and the outside.

As previously mentioned, in reference to the example movement detection of FIG. 3, the periods 300 to 304 can be determined for each room 20, 21 during the learning mode.

The monitoring system makes it possible to monitor several aspects related to each room 2. To that end, said secondary detection means 4 can comprise at least one detector of the environmental conditions of said room 2. Furthermore, non-limitingly, said environmental conditions comprise at least the temperature and/or the hygrometry. Then, said processing means apply at least one timestamp to the detected environmental conditions, so as to determine normal environmental conditions recorded within said monitoring profile.

In other words, aside from the monitoring of the movements of a person 1 within a room 2, the invention makes it possible to determine its normal use, by measuring and recording different parameters during a typical operation by said person 1. As an example, during the use of the kitchen, cooking food logically causes a temperature increase inside said room, during a variable duration depending on the dish being prepared, but often around the same time ranges (morning, afternoon and evening). During the use of the bathroom, morning and/or evening, the duration of a shower and/or a bath causes a significant increase in the temperature and the ambient hygrometry during use, which will next decrease once the shower is finished, when the person 1 dries off.

FIG. 2 shows an example of detection of the temperature and the hygrometry during the use of a room 2 like the bathroom. Over time according to the x-axis, the upper curve 40 shows the measured variations in the hygrometry, while the lower curve 41 shows the fluctuations in temperature. Several successive time periods 400 to 404 are determined, corresponding to the use already observed and recorded by the person within said room 2. These periods 400 to 404 can be determined by passing thresholds, above or below, depending on the evolution of the measured and recorded values of the increases and decreases in hygrometry and temperature. Each period may correspond to a behavior in the monitored room 2, like the bathroom.

Then, in comparative mode, said processing means perform a comparison of the detected environmental conditions with said recorded normal environmental conditions. Returning to the example of FIG. 2, if one of the curves 40, 41 departs from the values of the curves already recorded and/or the time ranges 400-404 already defined, then this logically results in an abnormal behavior, requiring an alert, locally or to the outside. For example, if the curve 40 of the hygrometry, at its peak showing the maximum, extends past the usual period 402, the person 1 may have fallen and cannot stop the shower: an outside intervention is then needed.

Furthermore, by combining the monitoring of the movements in environmental conditions, it is possible to further specify the behavior of the person 1 within said room 2. Returning to the two examples of FIGS. 2 and 3, the system is able to couple the measured and stored data, refining monitoring.

Multiple other examples can be considered, based on the monitored room 2 and types of sensors installed therein. In no way limitingly, other factors can be considered and measured, such as the brightness, the opening and closing of a closet/drawer/door/window, etc.

Alternatively, during the second duration being monitored, the measured data can be recorded similarly to the first learning duration, making it possible either to refine the profile of the person 2 and his behaviors, which may then be combined, in the form of an average with the profile already determined, or to determine at least one other profile, over a duration similar to the first duration, to compare it with the profile initially learned, in order to detect a potential deviation from the usage habits of each room 2 by the person 1. As an example, a significant extension of the time in the shower or delaying times where the person gets up may reflect a failure due to aging, requiring care in the short-, medium- or long-term, no longer at home but in a supportive environment.

The monitoring system according to the invention therefore makes it possible to determine, after a learning period, one or several profiles of a person 1 based on the typical use he makes of each room 2 of his home being monitored. These profiles make it possible to determine, based on the time and values of data making it up, whether a person 1 significantly changes behavior, by exceeding durations and time periods for each profile, as well as values showing an abnormal use of the room 2 subject to each profile.

1. A system for monitoring a person within at least one room, comprising:
at least separate primary and secondary detection means arranged inside said room, each detection means measuring data within said room and being comprised of means for sending data measured through a communication network; and
centralization means being comprised of means for receiving said data measured by each detection means, and means for processing the received measured data,
wherein the means for processing comprises at least one software application,
wherein the software application is executed in a learning mode over a first predefined duration, storing said measured and received data, so as to determine at least one monitoring profile of said person within each room, and
wherein the software application is executed in a comparative mode over a second duration, comparing said measured and received data with said stored data, so as to emit an alert signal if at least one of said data measured and received during said second duration deviates from an interval defined around said monitoring profile.

2. The monitoring system according to claim 1, wherein said primary detection means comprise at least one movement detector, determining the mobile presence of a person within said room, and wherein said processing means apply at least one timestamp to each determined presence, so as to define at least one presence time range of said person moving within said room.

3. The monitoring system according to claim 2, wherein, in learning mode, said processing means perform at least one weighted average of at least two time ranges defined successively, so as to determine, within said monitoring profile, at least one normal presence period within said room.

4. The monitoring means according to claim 1, wherein said secondary detection means comprise at least one detector for the environmental conditions of said room, said environmental conditions comprising at least the temperature and/or the hygrometry, and wherein said processing means apply at least one timestamp to the detected environmental conditions, so as to determine normal environmental conditions recorded within said monitoring profile.

5. The monitoring system according to claim 4, wherein, in comparative mode, said processing means perform a comparison of the detected environmental conditions with said recorded normal environmental conditions.
